(19) United States
(12) Patent Application Publication
(10) Pub. No.: US 2018/0193019 A1
(43) Pub. Date: Jul. 12, 2018

(54) BIDIRECTIONAL REVERSIBLE MEDICAL SKIN TENSION REDUCING CLOSER

(71) Applicant: HENAN HUIBO MEDICAL CO., LTD, Nanyang, Henan (CN)

(72) Inventors: Shuai YANG, Nanyang City (CN); Tiangang ZHU, Nanyang City (CN); Yao LIU, Nanyang City (CN); Xun LIU, Nanyang City (CN); Xinqin ZHOU, Pingdingshan City (CN)

(21) Appl. No.: 15/741,938

(22) PCT Filed: Jul. 19, 2016
(86) PCT No.: PCT/CN2016/090372

§ 371 Date: Jan. 4, 2018

(30) Foreign Application Priority Data
Jul. 26, 2015 (CN) 201510441898.0

(51) Int. Cl. A61B 17/08

(52) U.S. Cl. CPC A61B 17/085; A61B 2017/00407; A61B 2017/086

(57) ABSTRACT

A bidirectional reversible medical skin tension reducing closer is provided. The closer comprises a left substrate and a right substrate, both of which are configured to be horizontally and symmetrically arranged, a left pawl and a right pawl fixedly arranged on adjacent ends of the left and right substrates respectively, and a left rack and a right rack, one end of each of which is fixedly connected to the left and right pawls respectively in an up-down side-by-side arrangement. Ratchets on the left and right pawls tilt towards the left and right substrates respectively, and the other ends of the left and right racks pass through the space beneath the right and left pawls respectively and are engaged with ratchets provided on the bottom of the right and left pawls respectively. Adhesive layers for fixing the left and right substrates onto the surface of the skin are arranged on the bottom surfaces of the left and right substrates. According to the invention, forces can be uniformly applied from two sides, and the closure degree of the closer can also be freely adjusted.

### TECHNICAL FIELD

The present invention relates generally to a surgical instrument. More particularly, present invention relates to a bidirectional reversible medical skin tension reducing closer for reducing the tension in the skin around a surgical incision or wound.

### BACKGROUND OF THE INVENTION

Closure of a surgical incision or wound is the most common problem in surgery. Traditionally, suturing needles and sutures are used for the treatment of the skin in surgery. This brings about not only a new trauma to the skin but also infections of the surgical incision or wound, residues of foreign matter, delayed healing of the surgical incision or wound and the like. A number of patent applications have been filed for this. Chinese patent no. ZL 03117364.0 discloses a apparatus for closing a surgical incision or wound, which mainly comprises two base strips, with lock holes and racks on each, for disposal on the two sides of the wound. Engagement of the racks and the lock holes can make the surgical incision or wound closed. However, there are also some shortcomings about the structure of this apparatus that should not be neglected:
(1) when the rack is drew from one end, a counterforce is needed for the adhesive strip at the other end, but it is difficult for the forces to be uniformly applied from the two ends and thus the operation is inconvenient;
(2) according to the above-mentioned invention in which a principle of ratchet is utilized, the locking force of the latch can be only adjusted in an unidirectional manner and the adjustment direction can not be reversed, so once the force is applied excessively, it may cause the surgical incision or wound to close excessively and raise, and another new apparatus has to be provided, which may lead to great increase of medical costs;
(3) the apparatus is suitable for the surgical incision or wound only in a linear or approximately linear shape, however in clinical practice most surgical incisions or wounds are irregular, and this apparatus is not able to be applied to the incisions or wounds having a curvature; and
(4) nylon plastic of this apparatus with high hardness may bring discomfort to the patient, which is also needed to be considered. These above problems have been solved by some patents/patent applications in some ways, but fundamentally there is no big change. So, the need for a apparatus for closing a surgical incision or wound, which is suitable for the surgical incision or wound in a curved shape and freely adjusting the tension in the skin around the incision or wound and is also easy to use, is urgent.

### SUMMARY OF THE INVENTION

Accordingly, it is an object of the invention to provide a skin tension reducing closer without suturing which is suitable for application of forces from two sides, free adjustment of the closure degree and various skin wounds.

A bidirectional reversible medical skin tension reducing closer of the present invention includes a left substrate and a right substrate, both of which are configured to be horizontally and symmetrically arranged, a left pawl and a right pawl fixedly arranged on adjacent ends of the left and right substrates respectively, and a left rack and a right rack. One end of each of the left and right racks is fixedly connected to the left and right pawls respectively in an up-down side-by-side arrangement. Ratchets on the left and right racks tilt towards the left and right substrates respectively, and the other ends of the left and right racks pass through the space beneath the right and left pawls respectively and are engaged with ratchets provided on the bottom of the right and left pawls respectively. Adhesive layers for fixing the left and right substrates onto the surface of the skin are arranged on the bottom surfaces of the left and right substrates.

A plurality of vent holes may be provided in the left and right substrates, and glue in the adhesive layers may be hypoallergenic. These can effectively make the skin covered by the substrates breathe freely and prevent the skin from ulceration.

The ends of the left and right racks positioned in guide slots of the right and left substrates respectively may be each provided with a head with a lateral-striped or frosted top surface.

The outer end of the each pawl may be configured to be an upwardly bent, upturned eaves structure.

Materials of the left and right substrates may include one of, or combinations of two or more of polymer materials such as nylon, polyethylene, polypropylene, polyvinyl chloride, or metal materials.

Specifically, the bidirectional reversible medical skin tension reducing closer of the invention includes two same substrates, each of which is provided with vent holes, an adhesive layer, a pawl and a rack. The adhesive layer is provided on the bottom of the substrate. The pawl is provided on the head of the substrate. In use, the substrates are adhered onto the two sides of a incision or wound, and then heads of the racks are drew so as to make tissue edges of the wound beneath the substrates brought together. When the racks are overdrew due to an excessive force, the pawls on the racks may be pulled to freely adjust the closure degree. According to the invention, forces can be uniformly applied from two sides, and the closure degree of the closer can be freely adjusted.

The principle and advantages of the invention will be described below in detail. The closer of the invention includes two same substrates, each of which is provided with vent holes, an adhesive layer, a pawl and a rack. The adhesive layer is provided on the bottom of the substrate. The pawl is provided on the head of the substrate.
(1) in use, the substrates are adhered onto the two sides of a wound, and then the racks are drew so as to make tissue edges of the wound beneath the substrates brought together.
(2) the outer end of each pawl has an upwardly bent, upturned eaves slope, thus forming a gap between the two pawls combined and the wound to prevent the coverage of the wound. Most of all, when the racks are overdrew due to an excessive force, the pawls of the racks may be pulled to freely adjust the closure degree. According to the invention, forces can be uniformly applied from two sides, and the closure degree of the closer can be freely adjusted.
(3) studies have found that the larger the tension in the skin on the sides of a sutured wound is, the less likely the tissue edges are brought together closely. Then, the patient's body may receive a message that proliferating tissue is needed. More scar tissue may grow to close the wound. After a long period, a visible scar like a centipede may be formed. Otherwise, when the tension in the skin on the sides of the sutured wound is much smaller or absent, the tissue edges may be brought together closely. Then, the message that proliferating tissue is needed, received by the patient's body, may be not strong. Only less tissue may be needed to grow to close the wound. Of the scars formed by this, most are linear. So, the tension in the skin on the sides of the wound can be effectively reduced so as to form an linear wound.
(4) it has been reported in literatures that reduction of the tension in the skin on the sides of the wound may be useful not only for the healing of the wound but also for the treatment for the scar newly formed. The scar tissue may shrink or be relieved effectively by the use of the closer according to the invention in conjunction with dressings, such as medical silicone gels, which can make the scar shrink.

In summary, the bidirectional reversible medical skin tension reducing closer of the invention may be applied to various surgical incisions or wounds and the treatment of various scars, producing a huge advantageous effect.

### BRIEF DESCRIPTION OF THE DRAWINGS

FIG. 1 is a schematic view of an exemplary embodiment of the invention;

FIG. 2 is a sectional view of FIG. 1;

FIG. 3 is an enlarged view of section A in FIG. 2;

FIG. 4 is a schematic view of a pawl opened;

FIG. 5 is a schematic view of closers according to the invention being applied to a linear wound;

FIG. 6 is a schematic view of the closers according to the invention being applied to a curved wound; and

FIG. 7 is a schematic view of the closers according to the invention being applied to a large wound (without the non-woven adhesive layer on the top of the closers).

### DETAILED DESCRIPTION OF THE INVENTION

The present invention will be described below in detail with reference to the accompanying drawings.

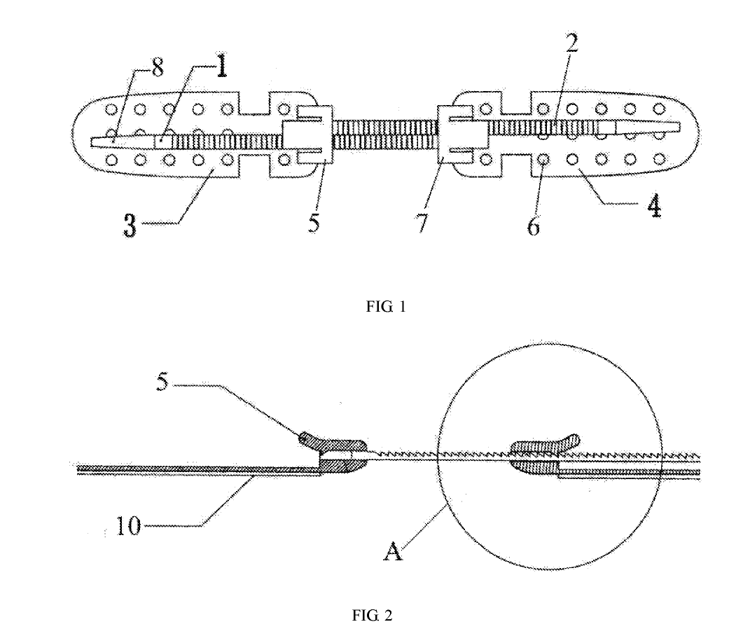

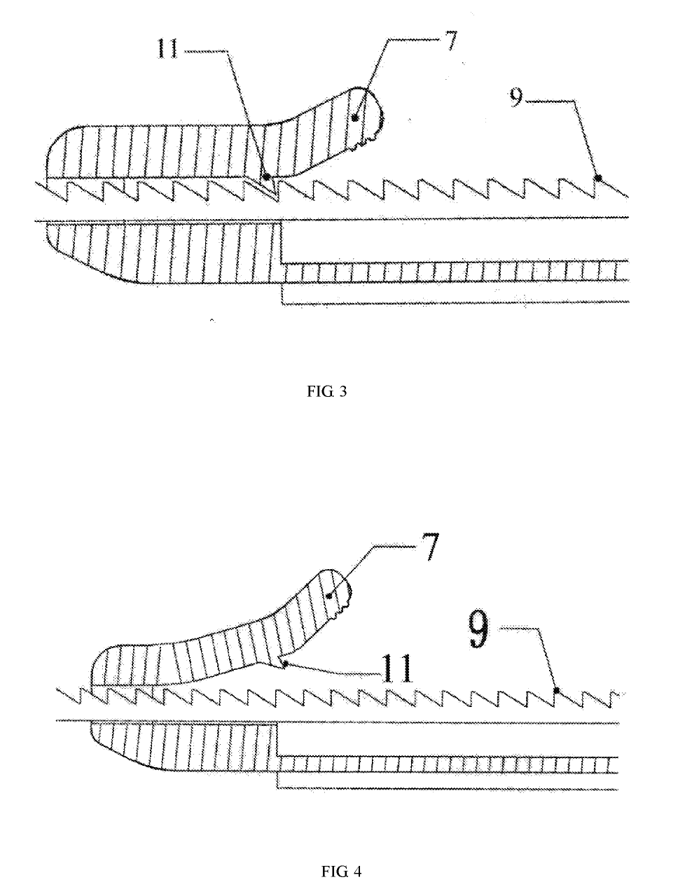

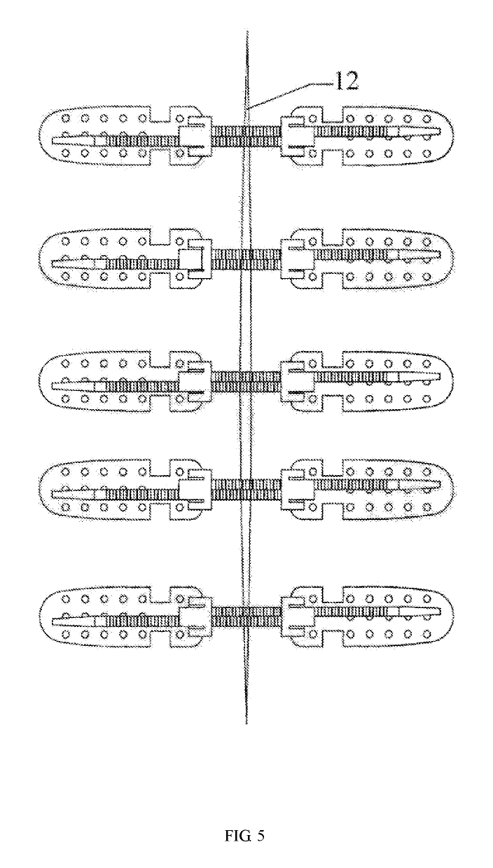

With reference to FIGS. 1 through 3, a bidirectional reversible medical skin tension reducing closer of the present invention includes a left substrate 3 and a right substrate 4, both of which are configured to be horizontally and symmetrically arranged, a left pawl 5 and a right pawl 7 fixedly arranged on adjacent ends of the left and right substrates respectively, and a left rack 2 and a right rack 1. One end of each of the left and right racks is fixedly connected to the left and right pawls respectively in an up-down side-by-side arrangement. Ratchets 9 on the left and right racks tilt towards the left and right substrates respectively, and the other ends of the left and right racks pass through the space beneath the right and left pawls respectively and are engaged with ratchets 11 provided on the beneath of the right and left pawls respectively. Adhesive layers 10 for fixing the left and right substrates onto the surface of the human skin are arranged on the bottom surfaces of the left and right substrates.

A plurality of vent holes 6 may be provided in the left and right substrates 3, 4, and glue in the adhesive layers 10 may be hypoallergenic. These can effectively make the skin covered by the substrates breathe freely and prevent the skin from ulceration. Further, if the wound in the injured area is large and the closers of the invention are not able to be fixed on the skin only by adhering it, then the closers may be fixed in the following way: the closers may be directly adhered onto the two sides of the wound, and then a skin stapler or a surgical suture may be used to make the substrates fixed on the two sides of the wound through the vent holes 6 in the substrates so as to make the closers fixed tightly.

The ends of the left and right racks positioned in guide slots of the right and left substrates respectively are each provided with a head 8 with a lateral striped or frosted top surface.

The outer end of the each pawl is configured to be an upwardly bent, upturned eaves structure.

Materials of the left and right substrates include one of, or combinations of two or more of nylon, polyethylene, polypropylene, polyvinyl chloride or metal.

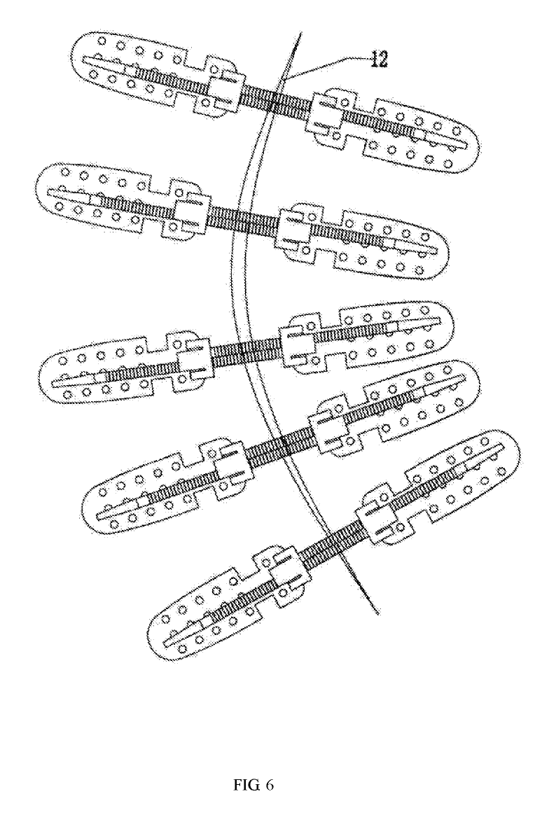

FIGS. 5 and 6 illustrate the closers of the invention being applied to a linear and a curved wound respectively. In use, the substrates are adhered onto the two sides of the wound, and then the racks are drew so as to make the tissue edges of the wound under the substrates brought together. When the racks are overdrew due to an excessive force, the pawls on the racks may be pulled to freely adjust the closure degree. According to the invention, forces can be uniformly applied from two sides, and the closure degree of the closer can be freely adjusted.

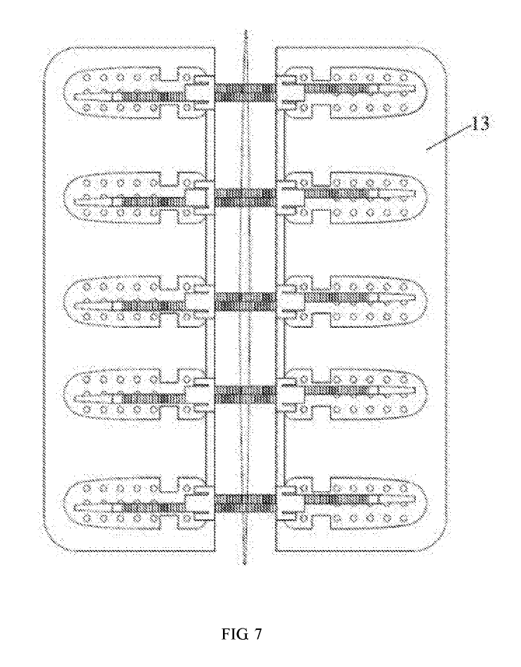

As shown in FIG. 7, when a large wound is treated, the closers of the invention may be symmetrically arranged and adhered onto the above non-woven adhesive layer, and then another non-woven adhesive layer is adhered onto the closers on the same side so as to make every single closer on the same side combined together via the two non-woven adhesive layers on the top of and at the bottom of the closers, forming an integral shape and thus assuring high fastness and stability among the closers.

### REFERENCE LIST

1 Right rack
2 Left rack
3 Left substrate
4 Right substrate
5 Left pawl
6 Vent hole
7 Right pawl
8 Rack head
9 Ratchet
10 Adhesive layer
11 Ratchet
12 Surgical incision or wound
13 Non-woven adhesive layer

1. A bidirectional reversible medical skin tension reducing closer, comprising:
a left substrate and a right substrate, both of which are configured to be horizontally and symmetrically arranged;
a left pawl and a right pawl fixedly arranged on adjacent ends of the left and right substrates respectively; and
a left rack and a right rack, one end of each of which is fixedly connected to the left and right pawls respectively in an up-down side-by-side arrangement;
wherein, ratchets on the left and right racks tilt towards the left and right substrates respectively, the other ends of the left and right racks pass through the space beneath the right and left pawls respectively and are engaged with ratchets provided on the bottom of the right and left pawls respectively, and adhesive layers for fixing the left and right substrates onto the surface of the skin are arranged on the bottom surfaces of the left and right substrates.

2. The closer of claim 1, wherein a plurality of vent holes are provided in the left and right substrates.

3. The closer of claim 1, wherein the ends of the left and right racks positioned in guide slots of the right and left substrates respectively are each provided with a head with a lateral-striped or frosted top surface.

4. The closer of claim 1, wherein outer ends of the left and right pawls are each configured to be an upwardly bent, upturned eaves structure.

5. The closer of claim 1, wherein materials of the left and right substrates comprise one of, or combinations of two or more of nylon, polyethylene, polypropylene, polyvinyl chloride, or metal.
